(19) United States
(12) Patent Application Publication
(10) Pub. No.: US 2023/0030417 A1
(43) Pub. Date: Feb. 2, 2023

(54) DATA COLLECTION DEVICE

(71) Applicant: Cheng Uei Precision Industry Co., LTD., New Taipei City (TW)

(72) Inventor: SHEN-TA TSAI, New Taipei City (TW)

(21) Appl. No.: 17/737,980

(22) Filed: May 5, 2022

(30) Foreign Application Priority Data
Jul. 27, 2021 (CN) 202121724415.5

(51) Int. Cl. G01N 27/04; G01N 33/24

(52) U.S. Cl. CPC G01N 27/048; G01N 33/24; G01N 2033/245

(57) ABSTRACT

A data collection device includes a main control module, at least one sensing module and a battery module. The main control module has at least one first connecting portion and at least one second connecting portion. The at least one sensing module is connected to the main control module. The at least one sensing module has a first conducting portion, an insulating layer and a second conducting portion. The insulating layer surrounds the first conducting portion. The second conducting portion surrounds the insulating layer. A top of the first conducting portion forms a first contacting portion. A bottom of the first conducting portion protrudes downward to form a second contacting portion. The first contacting portion is connected to the at least one first connecting portion. A top of the second conducting portion is connected to the at least one second connecting portion.

### CROSS-REFERENCE TO RELATED APPLICATION

The present application is based on, and claims priority from, China Patent Application No. 202121724415.5, filed Jul. 27, 2021, the disclosure of which is hereby incorporated by reference herein in its entirety.

### BACKGROUND OF THE INVENTION

### 1. Field of the Invention

The present invention generally relates to a data collection device, and more particularly to a data collection device capable of sensing environment states.

### 2. The Related Art

In traditional agriculture production, crops are affected by a climate and experiences of farmers, correspondingly, production quality of the crops are affected. The climate and the experiences of the farmers have unstable and sufficiently objective indexes, consequently, detail technologies of production are hardly inherited, and then the production quality of the crops are affected. Therefore, a conventional data collection device of soils is used to detect conditions of the soils.

When the conventional data collection device measures humidity of the soil, a measuring method is described as follows. The conventional data collection device uses at least two metal probe elements to be inserted into the soil, the humidity of the soil is calculated via an electric conductivity which is sensed by the at least two metal probe elements.

However, this measuring method requires the at least two metal probe elements, the conventional data collection device is able to analyze and calculate the humidity of the soil, so the conventional data collection device has a larger volume in use.

Thus, it is necessary to provide an innovative data collection device, the innovative data collection device is capable of sensing environment states, the innovative data collection device is able to measure the conditions of the soils with at least one single metal probe element to reduce a total volume of the innovative data collection device, so the innovative data collection device has a smaller volume in use.

### SUMMARY OF THE INVENTION

An object of the present invention is to provide a data collection device. The data collection device includes a main control module, at least one sensing module and a battery module. The main control module has at least one first connecting portion and at least one second connecting portion. The at least one sensing module is connected to the main control module. The at least one sensing module is a probe element. The at least one sensing module has a first conducting portion, an insulating layer and a second conducting portion. The first conducting portion and the second conducting portion are made of two different metal materials. The insulating layer surrounds a periphery of the first conducting portion. The second conducting portion surrounds a periphery of the insulating layer. A top of the first conducting portion forms a first contacting portion exposed upward from a top of the insulating layer. A bottom of the first conducting portion protrudes downward to form a second contacting portion exposed from a bottom of the insulating layer. The first contacting portion is connected to the at least one first connecting portion. A top of the second conducting portion is connected to the at least one second connecting portion. The second contacting portion and the second conducting portion are in contact with an object under test. The battery module is connected to the main control module.

Another object of the present invention is to provide a data collection device. The data collection device includes a main control module, an auxiliary control module, at least one sensing module and a battery module. The auxiliary control module is connected to the main control module. The auxiliary control module is provided with at least one first connecting portion and at least one second connecting portion. The at least one sensing module is connected to the auxiliary control module and the main control module. The at least one sensing module is a probe element. The at least one sensing module has a first conducting portion, an insulating layer and a second conducting portion. The first conducting portion and the second conducting portion are made of two different metal materials. The insulating layer surrounds a periphery of the first conducting portion. The second conducting portion surrounds a periphery of the insulating layer. A top of the first conducting portion forms a first contacting portion exposed upward from a top of the insulating layer. A bottom of the first conducting portion protrudes downward to form a second contacting portion exposed from a bottom of the insulating layer. The first contacting portion is connected to the at least one first connecting portion. A top of the second conducting portion is connected to the at least one second connecting portion. The second contacting portion and the second conducting portion are in contact with an object under test. The battery module is connected to the main control module.

Another object of the present invention is to provide a data collection device contacting with an object under test. The data collection device includes a main control module, an auxiliary control module, at least one sensing module, a battery module and a wireless transmission module. The auxiliary control module is connected to the main control module. The auxiliary control module is provided with at least one first connecting portion and at least one second connecting portion. The at least one sensing module is connected to the auxiliary control module and the main control module. The at least one sensing module is a probe element. The at least one sensing module has a first conducting portion, an insulating layer and a second conducting portion. The first conducting portion and the second conducting portion are made of two different metal materials. The insulating layer surrounds a periphery of the first conducting portion. The second conducting portion surrounds a periphery of the insulating layer. The first conducting portion is isolated from the second conducting portion by the insulating layer. A top of the first conducting portion forms a first contacting portion exposed upward from a top of the insulating layer. A bottom of the first conducting portion protrudes downward to form a second contacting portion exposed from a bottom of the insulating layer. The first contacting portion is connected to the at least one first connecting portion. A top of the second conducting portion is connected to the at least one second connecting portion. The second contacting portion and the second conducting portion are in contact with the object under test. The battery module is connected to the main control module. The wireless transmission module is connected to the main control module.

As described above, the insulating layer and the second conducting portion of the at least one sensing module of the data collection device surround the first conducting portion, the first conducting portion is isolated from the second conducting portion by the insulating layer, so the single sensing module which is the at least one metal probe element has two metal probes. Correspondingly, the data collection device is capable of sensing environment states in the same sensing module, the data collection device is able to measure conditions of soils with the at least one single metal probe element to reduce a total volume of the data collection device, so the data collection device has a smaller volume in use. As a result, a volume requirement of the data collection device is reduced, and a cost of the data collection device is saved.

### BRIEF DESCRIPTION OF THE DRAWINGS

The present invention will be apparent to those skilled in the art by reading the following description, with reference to the attached drawings, in which:

FIG. 1 is a perspective view of a data collection device in accordance with the present invention;

FIG. 2 is a system block diagram of the data collection device in accordance with the present invention;

FIG. 3 is another system block diagram of the data collection device in accordance with the present invention;

FIG. 4 is a cross-section view of the data collection device along a line IV-IV of FIG. 1; and

FIG. 5 is an enlarged view of an encircled portion V of the data collection device of FIG. 4.

### DETAILED DESCRIPTION OF THE PREFERRED EMBODIMENT

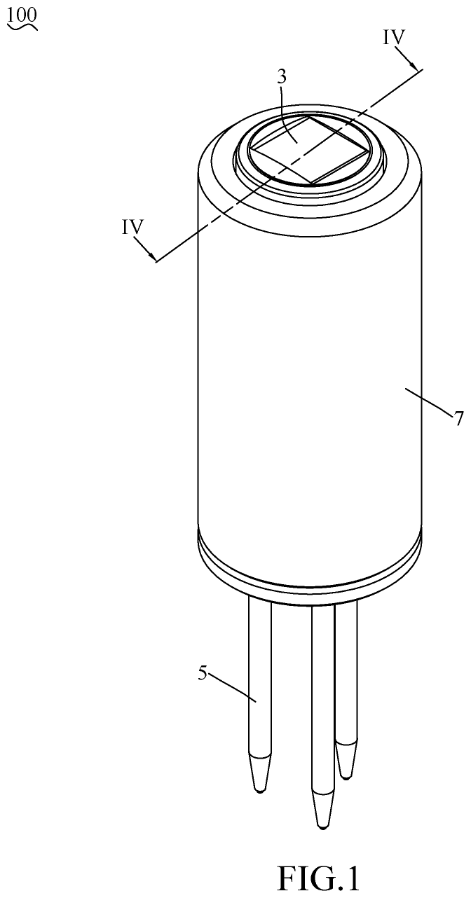

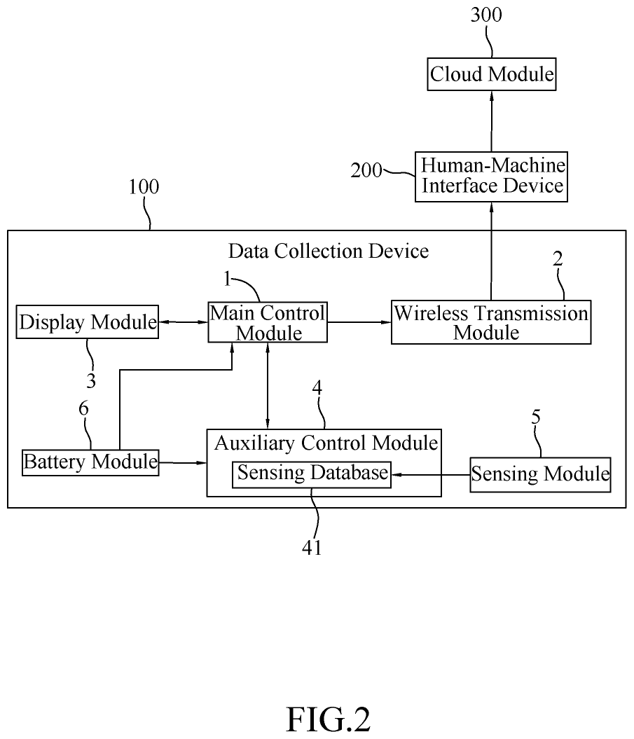

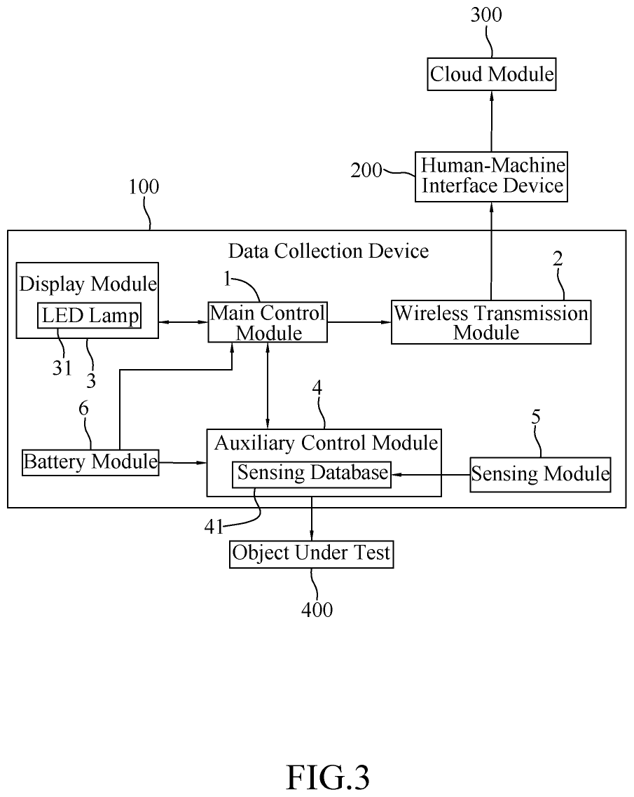

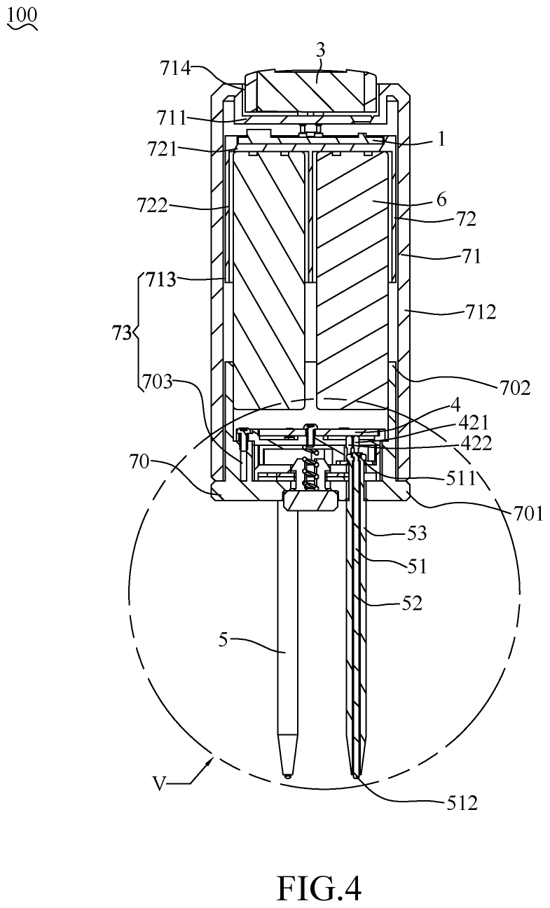

With reference to FIG. 1 to FIG. 4, a data collection device 100 in accordance with the present invention is shown. The data collection device 100 includes a main control module 1, a wireless transmission module 2, a display module 3, an auxiliary control module 4, at least one sensing module 5, and a battery module 6. The data collection device 100 contacts with an object under test 400.

The main control module 1 is a logical processor. The main control module 1 is used to proceed with various data calculations, such as temperature, humidity, potential of hydrogen (PH), time data calculations, data storage and other functions. The main control module 1 has at least one first connecting portion 421 and at least one second connecting portion 422.

The wireless transmission module 2 is connected to the main control module 1. The wireless transmission module 2 is able to receive various data of the main control module 1. The wireless transmission module 2 is connected to a human-machine interface device 200 through wireless transmission functions of the wireless transmission module 2 and the human-machine interface device 200. The wireless transmission functions of the wireless transmission module 2 and the human-machine interface device 200 are wireless network transmission functions or Bluetooth transmission functions, etc. The human-machine interface device 200 is an external device. The human-machine interface device 200 is able to receive the various data in the main control module 1 which is sent by the wireless transmission module 2. The various data in the main control module 1 is temperature, humidity, time and etc.

The human-machine interface device 200 is able to connect to a cloud module 300. The cloud module 300 is able to receive data from the human-machine interface device 200, and the data from the human-machine interface device 200 is stored in the cloud module 300.

The display module 3 is connected to the main control module 1. The display module 3 is able to display the data of the main control module 1 which is the temperature, the humidity, the time and a data storage. The display module 3 is a screen. The display module 3 is also provided with at least one LED (Light-Emitting Diode) lamp 31. The at least one LED lamp 31 is able to prompt the user to understand a current detection state.

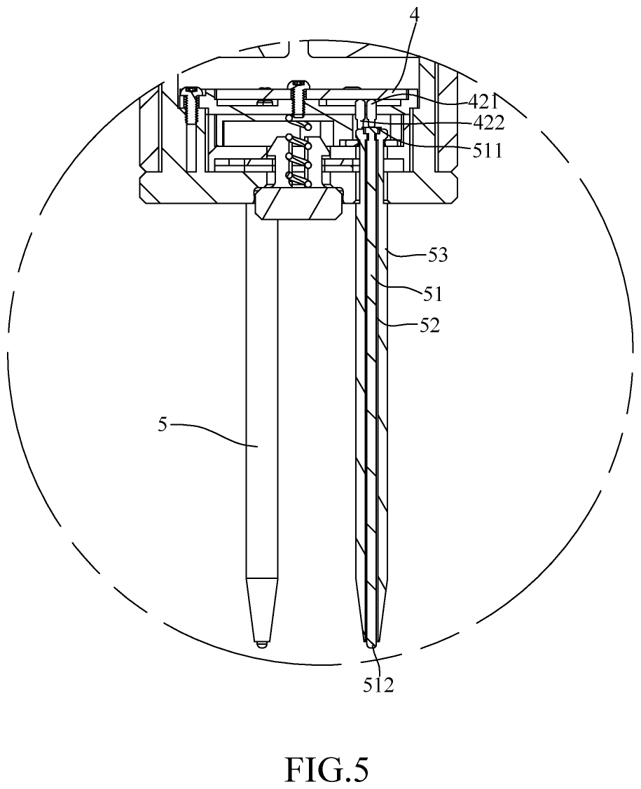

With reference to FIG. 1 to FIG. 5, the auxiliary control module 4 is connected to the main control module 1 and the battery module 6. The auxiliary control module 4 includes a sensing database 41. The sensing database 41 presets standard data, and the standard data is acted as a standard value of the data which is to be sensed. Specifically, the standard data is acted as the standard value for the temperature, the humidity, the potential of hydrogen (PH) and etc. The auxiliary control module 4 is provided with the at least one first connecting portion 421 and the at least one second connecting portion 422. The at least one first connecting portion 421 and the at least one second connecting portion 422 are mounted to the auxiliary control module 4. Both the at least one first connecting portion 421 and the at least one second connecting portion 422 are made of metal materials for conducting electrical signals.

The at least one sensing module 5 is connected to the auxiliary control module 4 and the main control module 1. The at least one sensing module 5 is able to be placed in a conductive medium to sense voltage values of an alkali metal and an alkaline earth metal of soils. A current voltage value sensed by the at least one sensing module 5 is transmitted to the sensing database 41, and the current voltage value sensed by the at least one sensing module 5 is compared with the preset standard data of the sensing database 41. Then the current voltage value is compared with the standard data to calculate data value of the currently sensed alkali metal and the currently sensed alkaline earth metal.

The at least one sensing module 5 is a probe element. Specifically, the at least one sensing module 5 is at least one metal probe element. The at least one sensing module 5 has a first conducting portion 51, an insulating layer 52 and a second conducting portion 53. The insulating layer 52 surrounds a periphery of the first conducting portion 51. The second conducting portion 53 surrounds a periphery of the insulating layer 52. The first conducting portion 51 is isolated from the second conducting portion 53 by the insulating layer 52, so the single sensing module 5 of the data collection device 100 which is the at least one metal probe element has two metal probes. A top of the first conducting portion 51 forms a first contacting portion 511 exposed upward from a top of the insulating layer 52. A bottom of the first conducting portion 51 protrudes downward to form a second contacting portion 512 exposed from a bottom of the insulating layer 52. The first contacting portion 511 is connected to the at least one first connecting portion 421 of the auxiliary control module 4. A top of the second conducting portion 53 is connected to the at least one second connecting portion 422 of the auxiliary control module 4. The second contacting portion 512 and the second conducting portion 53 are in contact with the object under test 400. In this way, the first conducting portion 51, the second conducting portion 53, the at least one first connecting portion 421 and the at least one second connecting portion 422 of the auxiliary control module 4 are in an electronic circuit. The at least one sensing module 5 is connected to the auxiliary control module 4.

The first conducting portion 51 and the second conducting portion 53 of the at least one sensing module 5 are made of two different metal materials. When the two different metal materials in the at least one sensing module 5 are electrically coupled to each other, and the two different metal materials in the at least one sensing module 5 are mutually conductive, the at least one sensing module 5 will generate a micro voltage characteristic. When the at least one sensing module 5 is inserted into the soils, the second contacting portion 512 and the second conducting portion 53 of the at least one sensing module 5 will be in contact with the conductive medium, and the voltage value of the at least one sensing module 5 will be changed. In this way, the at least one sensing module 5 gets the data value of the alkali metal and the alkaline earth metal of the soils.

When the voltage value of the at least one sensing module 5 changes, the at least one sensing module 5 transmits the voltage value to the auxiliary control module 4. The voltage value of the at least one sensing module 5 is compared with the standard data of the auxiliary control module 4 to calculate the data value of the alkali metal and the alkaline earth metal of the soils. Then the auxiliary control module 4 transmits the data value of the alkali metal and the alkaline earth metal of the soils to the main control module 1. The main control module 1 converts the data value of the alkali metal and the alkaline earth metal of the soils to humidity data. The main control module 1 transmits the humidity data to the display module 3 to display the current detection state, or the main control module 1 transmits the sensed data to the human-machine interface device 200 by the wireless transmission module 2, specifically, the main control module 1 transmits the humidity data to wireless transmission module 2 so as to make that the main control module 1 transmits the humidity data to the human-machine interface device 200 by the wireless transmission module 2.

The battery module 6 is connected to the main control module 1 and the auxiliary control module 4. The battery module 6 is used to provide electricity power for the main control module 1 and the auxiliary control module 4.

The data collection device 100 is normally in a standby state, sensing function of the at least one sensing module 5 and displaying function of the display module 3 are without working in the standby state of the data collection device 100. When the user wants to know one of the sensed data (such as the temperature, the humidity, or the time etc.), the user sends a signal through the human-machine interface device 200 to notify the data collection device 100. At the moment, the data collection device 100 starts working, the at least one sensing module 5 starts sensing the corresponding data, the currently sensed voltage value is transmitted to the sensing database 41, and then the currently sensed voltage value is compared with the preset standard data to calculate currently sensed data value. In this way, the user can know the data that the user wants to know, and the user saves energy expenditures of the data collection device 100.

The auxiliary control module 4 transmits the calculated data value to the main control module 1, and the calculated data value is stored in the main control module 1. The calculated data value is transmitted to the display module 3 through the main control module 1, and the calculated data value is displayed on the display module 3. Or the temporarily stored data value is transmitted to an external device by the wireless transmission module 2, specifically, the temporarily stored data value is transmitted to the human-machine interface device 200 by the wireless transmission module 2, so that the user is able to know the data which the user wants to know. A waking way according to a requirement is able to save an energy dissipation of the data collection device 100, and a calculation way is that the auxiliary control module 4 proceeds with a single sensing data, so that a calculation of the auxiliary control module 4 is simplified, and then needed hardware resources of the main control module 1 and the auxiliary control module 4 in calculations are saved.

Referring to FIG. 1 to FIG. 5, the data collection device 100 has a housing 7 enclosing the main control module 1, the wireless transmission module 2, the auxiliary control module 4 and the battery module 6. The housing 7 has a bottom housing 70, a top housing 71 surrounding the bottom housing 70, and an inner cradle 72 positioned inside the top housing 71. The bottom housing 70 has a bottom plate 701, an inner peripheral wall 702 extended upward from the bottom plate 701, and a bottom accommodating space 703 formed between the bottom plate 701 and the inner peripheral wall 702. The top housing 71 has a top plate 711, an outer peripheral wall 712 extended downward from the top plate 711, and a top accommodating space 713 formed between the top plate 711 and the outer peripheral wall 712. The top housing 71 further has a top cavity 714 formed on a top surface of the top plate 711. When the bottom housing 70 are assembled with the top housing 70, the bottom accommodating space 703 are communicated with the top accommodating space 713 to form an accommodating space 73. The inner cradle 72 has a middle plate 721, and at least one positioning portion 722 extended downward from the middle plate 721.

The auxiliary control module 4 is fixed in the bottom accommodating space 703 of the bottom housing 70. The main control module 1 is fixed in the top accommodating space 713 of the top housing 71. The main control module 1 is mounted to a top surface of the middle plate 721. The display module 3 is mounted in the top cavity 714 of the top housing 71. The battery module 6 is received in the accommodating space 73 of the housing 7. A top of the battery module 6 is fixed by the at least one positioning portion 722 of the inner cradle 72, and a bottom of the battery module 6 is fixed by the inner peripheral wall 702 of the bottom housing 70. The wireless transmission module 2 is an antenna which is directly formed on an inner surface of the top housing 71, an outer surface of the inner cradle 72 or an inner surface of a wall of the accommodating space 73. The antenna is able to be an independent component attached to the inner surface of the top housing 71, the outer surface of the inner cradle 72 or the inner surface of the wall of the accommodating space 73.

The display module 3 is mounted in the top cavity 714 of the top plate 711 of the top housing 71. The inner cradle 72 is received in the top accommodating space 713 of the top housing 71. The main control module 1 is mounted on the top surface of the middle plate 721 of the inner cradle 72. The at least one positioning portion 722 of the inner cradle 72 surrounds the top of the battery module 6. The bottom of the battery module 6 is received in the bottom accommodating space 703, and the bottom of the battery module 6 is surrounded by the inner peripheral wall 702 of the bottom housing 70. The auxiliary control module 4 is fixed in the bottom accommodating space 703 of the bottom housing 70, and the auxiliary control module 4 is positioned under the battery module 6. A top of the at least one sensing module 5 is received in the bottom accommodating space 703, and a bottom of the at least one sensing module 5 penetrates through the bottom plate 701 of the bottom housing 70 to be exposed outside of the housing 7. Therefore, a volume requirement of the data collection device 100 is reduced.

As described above, the insulating layer 52 and the second conducting portion 53 of the at least one sensing module 5 of the data collection device 100 surround the first conducting portion 51, the first conducting portion 51 is isolated from the second conducting portion 53 by the insulating layer 52, so the single sensing module 5 which is the at least one metal probe element has the two metal probes. Correspondingly, the data collection device 100 is capable of sensing environment states in the same sensing module 5, the data collection device 100 is able to measure conditions of the soils with the at least one single metal probe element to reduce a total volume of the data collection device 100, so the data collection device 100 has a smaller volume in use. As a result, a volume requirement of the data collection device 100 is reduced, and a cost of the data collection device 100 is saved.

What is claimed is:

1. A data collection device, comprising:
a main control module having at least one first connecting portion and at least one second connecting portion;
at least one sensing module connected to the main control module, the at least one sensing module being a probe element, the at least one sensing module having a first conducting portion, an insulating layer and a second conducting portion, the first conducting portion and the second conducting portion being made of two different metal materials, the insulating layer surrounding a periphery of the first conducting portion, the second conducting portion surrounding a periphery of the insulating layer, a top of the first conducting portion forming a first contacting portion exposed upward from a top of the insulating layer, a bottom of the first conducting portion protruding downward to form a second contacting portion exposed from a bottom of the insulating layer, the first contacting portion being connected to the at least one first connecting portion, a top of the second conducting portion being connected to the at least one second connecting portion, the second contacting portion and the second conducting portion being in contact with an object under test; and
a battery module connected to the main control module.

2. The data collection device as claimed in claim 1, further comprising a wireless transmission module, the wireless transmission module being connected to the main control module.

3. The data collection device as claimed in claim 1, further comprising an auxiliary control module, the auxiliary control module being connected to the main control module and the battery module, the auxiliary control module including a sensing database, the sensing database presetting standard data, the standard data being acted as a standard value of data which is to be sensed.

4. The data collection device as claimed in claim 3, wherein the auxiliary control module is provided with the at least one first connecting portion and the at least one second connecting portion, the at least one first connecting portion and the at least one second connecting portion are mounted to the auxiliary control module, the at least one sensing module is connected to the auxiliary control module.

5. The data collection device as claimed in claim 1, further comprising a display module, the display module being connected to the main control module, the display module being a screen.

6. The data collection device as claimed in claim 1, further comprising a display module, the display module being provided with at least one LED (Light-Emitting Diode) lamp.

7. The data collection device as claimed in claim 1, wherein the at least one sensing module is at least one metal probe element.

8. The data collection device as claimed in claim 1, wherein the first conducting portion is isolated from the second conducting portion by the insulating layer, so the single sensing module has two metal probes.

9. A data collection device, comprising:
a main control module;
an auxiliary control module being connected to the main control module, the auxiliary control module being provided with at least one first connecting portion and at least one second connecting portion;
at least one sensing module connected to the auxiliary control module and the main control module, the at least one sensing module being a probe element, the at least one sensing module having a first conducting portion, an insulating layer and a second conducting portion, the first conducting portion and the second conducting portion being made of two different metal materials, the insulating layer surrounding a periphery of the first conducting portion, the second conducting portion surrounding a periphery of the insulating layer, a top of the first conducting portion forming a first contacting portion exposed upward from a top of the insulating layer, a bottom of the first conducting portion protruding downward to form a second contacting portion exposed from a bottom of the insulating layer, the first contacting portion being connected to the at least one first connecting portion, a top of the second conducting portion being connected to the at least one second connecting portion, the second contacting portion and the second conducting portion being in contact with an object under test; and
a battery module connected to the main control module.

10. A data collection device contacting with an object under test, comprising:
a main control module;
an auxiliary control module being connected to the main control module, the auxiliary control module being provided with at least one first connecting portion and at least one second connecting portion;
at least one sensing module connected to the auxiliary control module and the main control module, the at least one sensing module being a probe element, the at least one sensing module having a first conducting portion, an insulating layer and a second conducting portion, the first conducting portion and the second conducting portion being made of two different metal materials, the insulating layer surrounding a periphery of the first conducting portion, the second conducting portion surrounding a periphery of the insulating layer, the first conducting portion being isolated from the second conducting portion by the insulating layer, a top of the first conducting portion forming a first contacting portion exposed upward from a top of the insulating layer, a bottom of the first conducting portion protruding downward to form a second contacting portion exposed from a bottom of the insulating layer, the first contacting portion being connected to the at least one first connecting portion, a top of the second conducting portion being connected to the at least one second connecting portion, the second contacting portion and the second conducting portion being in contact with the object under test;
a battery module connected to the main control module; and
a wireless transmission module connected to the main control module.
